(19) United States
(12) Patent Application Publication
(10) Pub. No.: US 2009/0202668 A1
(43) Pub. Date: Aug. 13, 2009

(54) Use of a polyphenol in the treatment of the metabolic syndrome and endothelial dysfunction or other vascular sequellae

(76) Inventor: Paul G. Hugenholtz, Oosterbeek (NL)

Correspondence Address: MILES & STOCKBRIDGE PC, 1751 PINNACLE DRIVE, SUITE 500, MCLEAN, VA, 22102-3833, US

(21) Appl. No.: 12/308,074

(22) PCT Filed: Jun. 7, 2007
(86) PCT No.: PCT/EP2007/005050

§ 371 Date: Mar. 9, 2009

(30) Foreign Application Priority Data
Jun. 7, 2006 (EP) 06076180.6

(51) Int. Cl. A61K 36/33; A61K 31/202; C07D 311/22

(52) U.S. Cl. 424/767; 514/560; 549/403

(57) ABSTRACT

The invention relates to the use of a polyphenol in the manufacture of a nutriceutical, a food, a food supplement or a medicament for the prophylactic or therapeutic treatment of endothelial dysfunction or the Metabolic Syndrome and other vascular sequellae. The invention further relates to a composition comprising at least one polyphenol and to a method for preparing such composition.

The invention relates to the use of a polyphenol in the treatment of endothelial dysfunction and/or of the Metabolic Syndrome or other vascular sequellae. The invention further relates to a composition comprising one or more polyphenols and to a method for preparing the components of such a composition.

It is generally accepted that cardiovascular disorders (endothelial dysfunction, the Metabolic Syndrome or other vascular sequellae, including obesitas, hypertension, atherosclerosis, diabetes) are the main causes of death and disease in the developed world. Primary prevention, rather than therapy once symptoms and signs have appeared, is the most logical approach and should, in principle, also provide the cheapest solution. Efforts in this direction by the World Health authorities and National Governments have met only with limited response and success.

However, it is not generally appreciated that the human body as it has evolved over the past 50,000 years with its diet of fish, fowl, game, berries, all products mainly found in nature, has been—rather “suddenly” in terms of the evolution duration—confronted around the year 1000 with newer foodstuffs such as grains, meat from domesticated animals and dairy products.

In the past 100 years this trend has exacerbated with refined derivatives such as blanched flour, pure sugar, rich dairy fats and red meats in which many essential nutrients were missing, artificially removed or altered to satisfy modern tastes rather than added or boosted to provide healthy, essential food components in order to maintain a normal nutritional balance.

It has been argued that in this relatively short time-span in the history of mankind, the human body has not had sufficient time to adapt to these sudden and rather drastic changes in modern nutrition. In fact, it is becoming evident that deficiencies in micronutrients have led to the appearance of diseases in the past century, such as beriberi (Vitamin B6) and scurvy (Vitamin C), when such substances were removed from daily nutrition.

A most striking example of nutritional maladaptation may be the onset of changes in the vascular system, rarely seen before 1900. The first target is the innermost layer of the human artery, the endothelium with a surface area of some 700 m² and a weight of ±2.5 kg. Since the Nobel prize winners Furchgott, Ignarro and Zawadzki elucidated the role of the healthy endothelium, we know that among its main functions are: the control of vascular wall tension, the maintenance of the correct balance between thrombosis and bleeding and the avoidance of vascular inflammation, all mediated through the adequate production of Nitric Oxide (NO).

Endothelial dysfunction has recently been shown to begin in early youth and over the ensuing years to lead to general vascular disease, ultimately culminating in atherosclerosis, hypertension and, eventually, heart, brain or kidney disease. The extent of this disorder is now epidemic, affecting millions of individuals most of whom are still in their early life and unaware of their affliction. The consequences of untreated endothelial dysfunction are detailed in P. Libby's 2002 seminal paper in the Scientific American: “Atherosclerosis, the New View” (Scientific American, 2002, May, p. 48-55). Indeed, endothelial dysfunction must now be thought of as a deficiency disease, like beriberi and scurvy in the recent past and therefore a solution must be sought for along similar lines of thinking.

Current medical therapeutical strategy in patients with (precursors of) vascular disease caused by endothelial dysfunction associated with abnormal cholesterol levels is mainly pharmaceutical and aims at lowering plasma LDL (Low Density Lipoprotein) levels to below 100 mg/l c.q. 2.0 mmol/l primarily by the ‘statin’ group of drugs such as 8-[2-(4-hydroxy-6-oxo-oxan-2-yl)ethyl]-3,7-dimethyl-1,2,3,7,8,8a-hexahydronaphthalen-1-yl]2-methylbutanoate. However, even in patients “optimally treated” cardiovascular events still do occur, while normocholesterolemic individuals may still acquire atherosclerosis.

Hence, increased interest by the pharma industry to develop compounds which increase HDL (High Density Lipoprotein), ‘the good cholesterol’. Current evidence (annual meeting of the American College of Cardiology, March 2007) that this can be achieved by pharmaceuticals is meagre and newer compounds such as the CETP-inhibitor Torcetrapib have shown poor results and major side effects such as death. The current state of affairs is extensively revealed in the 3^{rd} edition of the Netherlands Textbook on Vasculaire Geneeskunde, edited by Profs. Büller, Kastelein and Stroes of the Amsterdam Medical Center, 3^{rd} edition (2007), p. 1-405.

Therefore, new ways must be found to lower LDL and raise HDL simultaneously or find other ways to protect the endothelium from the inflammatory effects of oxygen radicals, oxidised LDL, etc.

Promising is the combination of protective nutritional components in a novel nutriceutical approach. Various groups of natural compounds present themselves: the polyphenols (Oligomeric Procyanidins, flavonoids, anthocyanidins, etc.) the omega-3 unsaturated fatty acids, and fatty acid binding fibres in the G.I. tract.

The inventors have come to the insight that at least part of the solution may lie in a radical rethinking of appropriate nutrition, mainly by the re-introduction of those elements from natural origin (“micronutrients”) which were gradually “forgotten” or eliminated by the food industry.

It is an object of the invention to provide a new treatment for a human in order to prevent the subject from developing a cardiovascular disorder, in particular the Metabolic Syndrome, or for a subject suffering from such disorder and its ultimate sequellae.

It is a further object of the invention to provide a novel composition, in particular a nutriceutical, that may be employed in the prophylactic or therapeutic use in a vascular disorder, such as the Metabolic Syndrome.

It is a further object of the invention to provide a method for preparing the components for such a novel composition, in particular a nutriceutical, suitable for treatment of a disorder, such as the Metabolic Syndrome and/or endothelial dysfunction or other vascular sequellae, in its various manifestations.

The inventors have found that it is possible to treat the Metabolic Syndrome and/or endothelial dysfunction or other vascular sequellae by a specific combination of components which are found in nature.

Accordingly, the present invention relates to the use of a polyphenol in the manufacture of a nutriceutical, a food, a food supplement or a medicament for the prophylactic or therapeutic treatment of the Metabolic Syndrome and/or endothelial dysfunction or other vascular sequellae.

In this document and its claims, reference to an element by the indefinite article ‘a’ or ‘an’ does not exclude the possibility that more than one element is present, unless the context clearly requires that there be one and one of the elements. The indefinite article ‘a’ or ‘an’ thus usually means at least one.

A “nutriceutical” is in particular a nutritional supplement designed for a clinical purpose.

The Metabolic Syndrome is a forerunner of generalized vascular disorders. The term “the Metabolic Syndrome” is used herein in agreement with the Adult Panel III of the National Cholesterol Education Program (NCEP). Herein it is defined that a subject suffers from The Metabolic Syndrome if at least three out of the following six symptoms can be diagnosed:
increased waist circumference (≧102 cm in men and ≧88 cm in women), indicating central obesity
elevated triglycerides (fasting triglyceride ≧69 mmol/l)
decreased HDL cholesterol (fasting HDL cholesterol <1.03 mmol/l for men, <1.29 mmol/l for women)
systolic blood pressure above 130 mm Hg and/or diastolic blood pressure above 85 mm Hg, and/or active treatment for hypertension
fasting glucose levels above 6.1 mmol/l and/or active treatment for hyperglycemia
recently was also suggested elevation of complement reactive protein (CRP)>1.6 mg/l.

The inventors have now found that a polyphenol which is obtainable from a fruit such as a grape is effective in the treatment of the Metabolic Syndrome

In a further aspect of the invention, one or more polyphenols are used in the prophylactic or therapeutic treatment of a (cardio)vascular disorder, wherein endothelial dysfunction together with endothelial destruction and plaque formation occur, a particular example being the (prophylactic) treatment of Acute Unstable Angina.

The inventors further have realised that a specific combination of a polyphenol and ω-3 fatty acids (in particular a combination of DHA and EPA) and/or NeOpuntia™ may show a synergistic effect.

A preferred product for use in accordance with the invention is Frutologic™ from the company BioSerae. This contains polyphenols from selected grapes and apples in a specific mixture

The efficacy of omega-3 fatty acids was recently confirmed in a large scale (randomized) clinical investigation comprising EPA+statin vs. statin alone in 18.645 patients with hypercholesterolemia by a 19% relative reduction in major coronary events (p=0.011), Lancet, 2007, vol. 369, p. 1090-1098.

NeOpuntia™ is a commercial name of a product (a cactus fibre) containing dehydrated cladodes of the Opuntia ficus-indica. In patent application WO 02/082930, of BioSerae Laboratoires, several methods are described to prepare powder particles of the cladodes of cactus Opuntia Ficus Indica, that are able to fix fats in the gastro intestinal tract. Where reference is made in this patent to ‘NeOpuntia™’, this includes all extracts such as obtained in accordance with the PCT application WO 02/082930, including extracts from other cactuses belonging to the Opuntioideae family. In particular it is contemplated that the combinations in accordance with the invention may exert a synergistic and corrective action on one or more vascular receptors which, when disturbed, lead to abnormal elevation of biomarkers in patients with the Metabolic Syndrome.

The primary mechanism of action of the omega 3 fatty acids [EPA+DHA] is the increase in NO formation with reduction in several inflammatory cytokines.

The primary mechanism of action of NeOpuntia™ lies in the binding of fatty food substances to the pulverized fibres of Opuntia ficus-indica leaves. The reduced availability of cholesterol in the caveolae results in a decrease in caveolin-1 at the cell surface of the endothelium in turn enhancing Nitric Oxide Synthase (NOS) activation. It is therefore hypothesized that the administration of NeOpuntia™ may amplify the NOS activation, induced by Frutologic™ itself.

In particular the use of a composition in accordance with the invention may have a positive (synergistic) effect on any one or more of the pro-atherogenic parameters, such as one or more indicative of inflammation, increased coagulation, excessive oxidation, elevated lipid parameters and generally disturbed endothelial function.

Although they do not wish to be bound by theory the inventors believe that these effects of polyphenols either alone or in combination with a combination of DHA and EPA and/or NeOpuntia™ can be explained by the action of all three components on one or more of the following biomarkers of the disturbed human circulation:
a. We have shown (see Vascular Protection by dietary polyphenols, Stocklet, J. C., et.al., Eur. J. Pharm. 2004, 500, 299-313-) that, the redox sensible P13-kinase dependent activation of endothelial NO synthase resulting in a long-lasting increased formation of nitric oxide (NO) and inhibition of endothelin (ET 1), a peptide which has a vasoconstricting effect, together result in vascular wall dilatation and improved systemic arterial compliance via the relaxations of vascular smooth muscle cells.

These in vitro studies with a red wine derived polyphenolic extract were repeated by Müller et al. (2005, unpublished data) with the product Frutologic™ which has a particular and specific mixture of multiple natural polyphenols in a concentration of 70%. Experiments on a rat in vitro model with amounts similar to and higher than our recommended dose of 250 mg twice a day (or 2 to 10 mg/kg/BW) for a human adult showed even higher degrees of vascular dilatation. Specific grapes, Merlot and San Giovese, exceeded even these. All experiments depended on an intact endothelium. Removal of the latter removed the dilatory response. When the dose of Frutologic™ increased to a level exceeding 30 mg/l, vascular contraction rather than dilation occurred. Comparable observations were made in early studies on biomarkers in a mice model which mimicked human atherosclerosis. These observations are consistent with isolated toxicity reports in humans after excessive ingestion (10-20 mg/kg/day) of Tea Vigo, a polyphenol preparation concentrated from green tea leaves. Dog studies have confirmed hepatotoxicity with this product. Accordingly we strictly recommend a dose of Frutologic™ of 250 mg twice daily which are proven effective in health clinics and sports centers in improving muscular performance. No side effects have been observed in a cohort of several hundred volunteers,
b. Thus therapy leads to a reduction c.q. normalization of the inflammatory biomarkers such as CRP, vascular cell adhesion molecule (V-CAM), endothelial cell adhesion molecule (E-CAM), platelet derived growth factor (PDGF), monocyte chemo attractant protein-I (MCP-1), interleukin 1 alpha (IL-1), tumour necrosis factor alpha (TNFα), among others. The normalization of the protection level by polyphenols, in particular from grapes, e.g. in the form of a Red Wine Polyphenol Concentrate (RWPC), such as extract of Merlot, Cabernet Sauvignon or San Giovese (Brancaia Winery) proving restoration of normal endothelial function and normal NO production among others via NO synthase stimulation, may be enhanced by DHA-EPA, as can be demonstrated by a study measuring a variety of inflammatory biomarkers.
c. Forearm blood flow and flow mediated dilation (FMD) may be normalised or restored as its central physiologic mechanism of action after the polyphenol (alone or in combination) has been administered, as has been demonstrated by a human study measuring these parameters
d. These effects may further be stimulated by reduction in thromboxane A2 production and diminished platelet aggregation through the action of blockage of platelet receptors and/or similar activity by DHA/EPA.
e. The antioxidant capacity of polyphenols via scavenging of free radicals and inhibition of pro oxidant enzymes such as NADPH-oxidase enhances blood flow and avoids platelet aggregation and vascular thrombosis.
f. The antioxidant capacity of polyphenols may also reduce the triglycerides oxidised Low Density Lipoprotein levels (oxLDL) and fibrinogen. Similarly, DHA, EPA, NeOpuntia™ and the polyphenols may synergistically reduce triglycerides so that the dangerous form of LDL, oxLDL, is blocked from causing endothelial dysfunction. HDL production is enhanced, providing a further separate protective system against oxLDL.
g. It is postulated that the effects under a, b, c, d, e and f exert a beneficial action on the severity and extent of endothelial damage, (partially) restoring normal endothelial function and thus reducing the increased vascular wall tension, in turn normalising blood flow and blood pressure throughout the human body, thereby exerting a primary preventive action against vascular disorders and their sequellae.
h. Furthermore, our experiments with polyphenols have shown that the levels of the cytokines, matrix metallo proteinase-II (MMP-II) as well as Vascular Endothelial Growth Factor (VEGF)—2 major proangiogenic growth factors—can be reduced. As these two cytokines will affect the stability and integrity of already existing atherosclerotic plaques within the endothelial dysfunctional area, by attacking the cap covering the plaque, the chance of plaque fissure or rupture will be diminished by the polyphenols. This action is exerted on the migration and proliferation of vascular cells thus reducing the extent of neoangiogenesis from the underlying adventitia. These actions will contribute to the normalisation of blood flow in an artery already affected by the disease process to such an extent that obstruction can occur. They will therefore extend the beneficial action of the polyphenols from a primary preventive mode (see g) to a secondary preventive mode. In other words not only avoiding endothelial dysfunction from becoming an atherosclerotic lesion but also reducing or eliminating already existing lesions and plaques. In other instances, just the stabilisation of the plaque cap may already constitute an effective therapy.

It is noted that compositions comprising polyphenols and/or omega-3 fatty acids or NeOpuntia™ are known in the art; however, clinical evidence of a prophylactic or therapeutic effect in diseased human beings has not been provided in a conclusive manner to date.

Although it has been suggested in general that red wine polyphenols could have multiple beneficial effects on arteries (see e.g. Red Wine Polyphenolic Compounds Inhibit Vascular Endothelial Growth Factor Expression in Vascular Smooth Muscle Cells by Preventing the Activation of the p38 Mitogen-Activated Protein Kinase Pathway, Oak e.a., Arterioscler Thromb Vasc. Biol. 2003; 23: 1001-1007) it is concluded that the full nature of the suggested effect is still unclear. It was also believed by scientists that the suggested positive effect of red wine may be contributed to ethanol (see e.g. Different effects of red wine and gin consumption on inflammatory biomarkers of atherosclerosis: a prospective randomized crossover trials Effects of wine on inflammatory markers, Estruch e.a., ATHEROSCLEROSIS; 175 (2004) 117-123. Estruch demonstrated in healthy volunteers that the polyphenols found in the wine extract and not in pure gin exerted a beneficial action. They concluded that their study is too short for any statements in relation to atherosclerosis.

Earlier, based on animal models, it has been suggested in the literature that polyphenols may improve endothelial function. Scientists however stress that due to absorption and metabolism of the polyphenols in vitro effects should always be confronted with in-vivo experiments and even clinical trials before it can be clearly stated that they underlie the protective properties in the cardiovascular system assigned to plant polyphenols. Which compounds are finally released in the circulation after ingestion of foods or beverages with complex polyphenolic compositions requires further investigations. It is suggested that the effects may depend on the type of active polyphenol, the dose and the extent of bioavailability.

In a recent study (Corder e.a., Red Wine procyanidins and vascular health, Nature, Vol 444, 30 Nov. 2006) it is confirmed that there is lack of consensus in science on the protective effects of red wine. It is suggested that this may be due to a variety of vasoactive constituents. Grape seeds are the main source of oligomeric procyanidins (OPC) but poor solubility and oenological and viticultural factors influence the amount of OPC's in wines. Again it is stressed that although absorption of OPC's has been demonstrated in vivo, little is known about their metabolic availability and metabolism.

On the other hand, Pigmatelli et al. “Polyphenols synergistically inhibit oxidative stress in subjects given red and white wine”, in Atherosclerosis, 2006, Vol 188, p. 77-83, have stressed that not only were plasma polyphenols higher after red wine compared to white wine, as measured by the excretion of isoprostanes in the urine (a breakdown product from prostaglandin F_PFG 2α-III) but also that subsequent in vitro analysis showed that only naturally occurring polyphenols, all in the range found from the in vivo study, exerted their anti oxidative activity. This was demonstrated by inhibition of LDL oxidation and proteinkinase-C (PKC) mediated NADPH oxidase activation. Earlier studies in our laboratory had already indicated that naturally occurring polyphenols—such as Frutologic™—were always more effective in terms of vascular dilatation than an artificial mixture of various prior known individual polyphenols, such as resveratol, catechins and caffeic acid. Such concoctions were always less effective than the naturally occurring extracts which were also active at lower concentrations suggesting synergistic action.

Very recently (2007) Greek Investigators (oral communication) have attempted to specify the effects of a specific grape (a rare Vinis Vitae) from the Aegean Island of Santorini. Their dealcoholized extract was given to patients with angiographicall proven coronary artery disease and symptoms of angina pectoris. This is the first direct application of a polyphenol extract in diseased humans with improvement in their vascular response as measured by (FMD).

The conclusion is that, due to the many factors influencing whether or not a given extract may have a vasoprotective effect, the only way to prove a possible therapeutic effect is by clinical trials in diseased human beings. The prior art, including the documents cited, therefore may suggest a beneficial effect but without evidence they cannot be considered as disclosing—with certainty and in a reliable manner—a composition containing at least a polyphenolic fraction in an amount to obtain a vasoprotective effect to prevent or treat a metabolic vascular disorder.

EP 1 640 001 (New Pharma Investments Holding) describes a multicomponent product, consisting of a combination of at least one vitamin, at least one soy derivative and a least one polyphenol. This product would be useful in preventing or treating cardiovascular diseases, including the metabolic syndrome. A multicomponent product was tested in an animal model. This document does however not disclose which components are necessary to achieve the relevant effect—no clinical trials are performed. This product has many disadvantages as it may contain ineffective components that may even be detrimental to the human body.

The same applies to the compositions disclosed in DE203 04 752 (Weber & Weber & Co. KG). This document merely suggests that polyphenols would have an antioxidant effect; however, the document does not suggest let alone disclose that the compositions or the polyphenolic fractions are indeed useful to prevent or treat people suffering from the metabolic syndrome.

WO 2005/099483 (EBN International Servicing Limited Liability Company) describes ingestible compositions for metabolic syndrome, but does not disclose the use of polyphenols or provide evidence of a positive effect.

WO 2004/087181 (Bioplanta Arzneimittel GmbH) discloses compositions containing combinations of a ω-3 fatty acid and a polyphenolic extract. The fatty acid preferably is α-linolenic acid. It is postulated that such composition may be used for the treatment of a variety of diseases, in particular various infections, immunological diseases, metabolic diseases or cancers. In this product the polyphenols are present to provide a protection for the increased need of the subject treated with the ω-3 fatty acid, because such fatty acid is highly oxidisation-sensitive.

EP-A 1 586 244 (Cognis IP Management GmbH) discloses a food composition comprising an ω-3 fatty acid and an antioxidant, such as a polyphenol. Herein the role of the polyphenol is to shield and protect the fatty acid from oxidation; an action at vascular level is not suggested nor claimed.

Therefore, the prior art cannot be considered as unambiguously disclosing the use of polyphenols for the treatment of the metabolic syndrome, nor will a person skilled in the art assume a reasonable expectation of success to be able to treat diseased human beings as the effect of polyphenols is, as indicated here before, dependent of many factors and circumstances.

A polyphenol can generally be defined as a compound having more than one phenol group per molecule. Over 500 polyphenols have been reported in the art. Polyphenols which may used in accordance with the invention in particular include polyphenols which may be found in specific grapes. Examples of polyphenols include flavones, isoflavones, anthocyanins, tannins, catechins, lignans, flavonoids, oligomeric proanthocyanidins (OPC).

As a source of polyphenols a plant extract is highly suitable. Preferably, a polyphenol extract from at least one fruit selected from grapes, berries (e.g. cranberries) and apples is used. A combination of polyphenolic compounds from grapes and from apples is in particular suitable.

Of the grapes, the Merlot, Cabernet Sauvignon and San Giovese are preferred. An extract of such grapes has a favourable polyphenolic profile. Particular suitable grapes of the type of Merlot include the clones of Merlot such as clones 181, 342, 343 and 348 obtainable from culture at the Brancaia Winery (Radda in Chianti, Italy).

Suitable ways of extracting a polyphenol from plant material are known in the art. Polyphenolic plant extracts are also commercially available. In literature it has been disclosed in November 2006 (Corder e.a., Red Wine procyanidins and vascular health, Nature, Vol 444, 30 Nov. 2006) for the first time that the correct OPC concentration may be one crucial factor in the vasoactivity of a polyphenolic extract and benefit. Total polyphenols correlated linearly with the suppression of ET-1 synthesis, a strong vaso-restrictive cytokin. the composition of which is consistently monitored by using techniques such as HPLC. Yet in their analyses of red wines from different European and World regions and the relationship to longevity (as an index of overall good health), those harvested from Nuoro (Italy) and the Gers region (France) with the unique flavonoid rich Tannat grape showed the greatest efficacy. A preferred product for use in accordance with the invention is Frutologic™ from the company BioSerae. This contains polyphenols from selected grapes and apples in a specific mixture

In order to obtain an extract demonstrating a high bioactivity in a preferred embodiment a polyphenol is extracted via a method comprising the following steps:
subjecting a fruit (such as grapes) to fermentation, in particular at a temperature of about 30-32° C.;
subjecting a fruit (a grape) to maceration and fermentation, in particular for a contact period of about 12-25 days (according to the wine maker's specification);
separating the maiche fraction from the liquid fraction; and

recovering a polyphenol from the liquid fraction, in particular by extraction.

It has been found that a high quality polyphenol is obtained by taking care that the fruit is (gently) compressed without damaging the pits and excluding the small branches before it is introduced into the fermentation reactor. Accordingly hand-picking of the fruit is preferred.

Other aspects of the production process that are considered advantageous with respect to obtaining a high quality polyphenol are the following.

It is advantageous to process the fruits quickly from picking to introduction into the fermentation reactor.

Fruits are preferably destemmed and lightly crushed prior to introduction into the fermentation reactor. The introduction preferably takes place without using a pump. In particular the ((partially) crushed) fruits are preferably allowed to slide into the reactor (making use of gravity).

The design of the reactor (such as a steel tank) is also found to be of relevance to the quality of the polyphenols. Preferably a conical reactor. is used. Such design enlarges the space of contact between grapes and must while reducing must-air contact.

During maceration the must is preferably moved by a pestle, to improve contacting of a fruit skins with the liquid. The moving is suitably is carried out at least 3 times a day, in particular 3-5 times a day. This helps to recover a polyphenol from the skin of the fruits.

It is contemplated that any of these steps/precautions alone or in any combination contribute to the quality of the polyphenol extract, in that they contribute to reduced oxidation of the polyphenols, or that they enhance their concentration.

DHA and EPA may in particular and preferably be obtained from a marine animal, such as fish. DHA, EPA and optionally one or more other ω-3 polyunsaturated fatty acids may be used as a free acid, as a salt thereof, as a chemically bound form, in particular an ester (such as mono-, di- or triglyceride) or in any combination thereof. Preferably DHA and EPA is present in a composition (used) according to the invention in a form selected from the group consisting of alkyl esters—in particular ethyl esters and free acids.

Suitable ways to provide the ω-3 polyunsaturated fatty acids are known in the art. Fish oil comprising [DHA and EPA] is commercially available. A suitable commercially available product comprising [DHA and EPA] is RoPuFa™ (supplier: DSM). The amount of ω-3 polyunsaturated fatty acids, such as [DHA and EPA], may be determined by gas chromatography.

For NeOpuntia™, recent studies have suggested that this product has a positive effect on blood lipid parameters and the cell membrane associated with cardiovascular risk, and thus prevent and/or treat the metabolic syndrome synergistically. The mechanism of action is presumed to be binding of fatty food substance to the fibres of the cactus referred to above.

One such study was recently completed by our group (unpublished data). It demonstrated in 59 women with the Metabolic Syndrome (ages 20-55), in a randomized placebo controlled, double blind study, that 1.6 gram of NeOpuntia™ (a product from dehydrated powdered Opuntia ficus indica) taken three times daily, over a period of only 6 weeks, reduced the symptoms and signs of the 5 criteria for Metabolic Syndrome (defined as HDL cholesterol≦0.5 g/l, Triglycerides≧1.49 g/l, Bloodpressure≧130/85 mmHg, Fasting glucose≧0.95 g/l and waist circumference≧80 cm) in 39% of the tested group vs. 8% in the controls. Most striking was the rise in HDL by 7% in the 42 females older than 45, or in those whose cholesterol at entry was ≧2.4 g/l. This efficacy is similar to that reached by statins. The combination of NeOpuntia™ with a product such as Frutologic™ will reinforce the action of the latter, by reducing cholesterol and fatty acid uptake and elevating HDL levels, thus enhancing the protective and therapeutic effects of the polyphenol product with or without the added benefit of omega-3 rich products containing hexanoic and eicosopentaenoic acid in the recommended doses. This synergistic effect is greater than the actions of each of the individual compounds alone.

The ratio of the sum of DHA plus EPA to a polyphenol is preferably in the range of 0.5-12. A composition comprising such ratio is considered particularly effective. The ratio of NeOpuntia™ to a polyphenol is preferably in the range of 0.5-12.

Accordingly, the present invention further relates to a nutriceutical, food supplement or pharmaceutical composition, comprising a polyphenol, in combination with [DHA and EPA] or with NeOpuntia™, wherein the weight to weight ratio of the sum of docosahexanoic acid and eicosopentaenoic acid, or NeOpuntia™, to polyphenol is in the range of 0.5-12.

In particular the ratio [DHA+EPA] to polyphenol(s) may be at least 1, more in particular at least 2.

In particular the ratio [DHA+EPA] to polyphenol(s) may be up to 8, more in particular up to 6, even more in particular up to 3.

The weight to weight ratio docosahexanoic acid to eicosopentaenoic acid is usually in the range of 1:10 to 5:1.

The [EPA and DHA] or NeOpuntia™ and polyphenol are preferably administered to provide a daily dosage of 500-3000 mg [EPA+DHA] or 5000 mg NeOpuntia™, and 250-500 mg polyphenol(s). In a highly preferred embodiment, the daily dosage of [EPA+DHA] (used) in accordance with the invention is up to 1500 mg, in view of a high clinical efficacy. For NeOpuntia™ the recommended dose is three times 1600 mg per day with meals.

The polyphenols(s), either alone or in combination with [DHA and EPA] or NeOpuntia™ may be administered in any nutriceutical or pharmaceutical form, including, but not limited to capsules, tablets, food supplements, lozenges or drinks.

In addition to [DHA and EPA], NeOpuntia™ and polyphenol one or more other active ingredients may be present, such as one or more other polyunsaturated fatty acids and/or one or more other antioxidants. In particular one or more other ω-3 polyunsaturated fatty acids may be present. It is preferred though that [DHA and EPA] provide at least 64% of the ω-3 fatty acids.

A composition (used) according to the invention may be essentially free of further active ingredients, or at least free of other active ingredients than those found in the sources for [DHA and EPA] or NeOpuntia™ and/or polyphenol such as one or more other polyunsaturated fatty acids found in a fish oil which may be used as a source for DHA and EPA and/or organic compounds, such as organic acids, found in the source for a polyphenol such as a fruit extract, in particular a RWPC. In particular a composition used in accordance with the invention may be essentially free of added vitamins and/or added amino acids

In addition to the active ingredients, it may comprise one or more excipients. Suitable excipients are known in the art. The polyphenols(s) either alone or in combination with [DHA+EPA] and/or NeOpuntia™ may conveniently be administered, as such or as a mixture (in the same dosage unit) or as a combination of separate dosage units (such as separate capsules, tablets, etc).

It is also possible to administer the polyphenols, either as such or in combination with [DHA and EPA] and/or Neopuntia™ in the form of a food, such as a dairy products, e.g. yoghurt, bread, margarine, soft drink or a juice. Subjects who may benefit from a use (of a composition) in accordance with the invention include any (diseased) humans, including adults and children, in particular adults and children from age 10 and older. The use (of a composition) in accordance with the invention may extend to one or more weeks, months or years.

1. Use of a polyphenol in the manufacture of a nutriceutical, a food, a food supplement or a medicament for the prophylactic or therapeutic treatment of Endothelial dysfunction or the Metabolic Syndrome or other vascular sequellae.

2. Use according to claim 1 wherein the polyphenol is obtained from fruit.

3. Use according to claim 1 in which the polyphenol is obtained from grapes.

4. Use according to claim 2 in which the fruit is a combination of grapes and apples.

5. Use according to claim 1, in which in addition either a combination of docosahexanoic acid and eicosopentaenoic acid, or dehydrated powder of Opuntia ficus indica is used.

6. Use according to claim 5, wherein the weight to weight ratio of the sum of docosahexanoic acid and eicosopentaenoic acid, or dehydrated powder of Opuntia ficus indica to polyphenol(s) is in the range of 0.5-12.

7. Use according to claim 5, wherein the weight to weight ratio of docosahexanoic acid to eicosopentaenoic acid is in the range of 1:10 to 5:1.

8. Use according to claim 1, wherein the nutriceutical, the food the food supplement or the medicament is to be administered to provide a daily dosage of 250-500 mg polyphenol(s).

9. Method for preparing a polyphenol for use in the manufacture of a nutriceutical, a food, a food supplement or a medicament for the prophylactic or therapeutic treatment of Endothelial dysfunction or the Metabolic Syndrome or other vascular sequellae, comprising:
subjecting the fruit to fermentation, for a period of about 12-25 days thereby forming a liquid fraction and a maiche fraction;
separating the maiche fraction from the liquid fraction; and
recovering polyphenols from the liquid fraction, by extraction.

10. (canceled)

11. Nutriceutical, food supplement or pharmaceutical composition comprising a polyphenol, optionally in combination with a combination of EPA+DHA and/or dehydrated powder of Opuntia ficus, for treatment or prevention of endothelial dysfunction or metabolic vascular disorder.

12. Composition according to claim 11, in which the polyphenols are obtained from fruit.

13. A polyphenol for use in the manufacture of a nutriceutical, a food, a food supplement or a medicament for the prophylactic or therapeutic treatment of Endothelial dysfunction or the Metabolic Syndrome or other vascular sequellae, obtainable by the method of claim 9.

14. Use according to claim 2, in which in addition either a combination of docosahexanoic acid and eicosopentaenoic acid, or dehydrated powder of Opuntia ficus indica is used.

15. Use according to claim 3, in which in addition either a combination of docosahexanoic acid and eicosopentaenoic acid, or dehydrated powder of Opuntia ficus indica is used.

16. Use according to claim 3, wherein the grapes are selected from the group consisting of Cabernet Sauvignon, Merlot, and San Giovese.

17. Use of a polyphenol in the manufacture of a nutriceutical, a food, a food supplement or a medicament for the prophylactic or therapeutic treatment of Endothelial dysfunction or the Metabolic Syndrome or other vascular sequellae wherein the polyphenol is obtained from fruit according to the method of claim 9.

18. Method according to claim 9, wherein subjecting the fruit to fermentation is at a temperature of about 30-32° C.

19. Method according to claim 9, wherein the fruit includes grapes.

20. Method according to claim 19, wherein the grapes are selected from the group consisting of Merlot, Cabernet Sauvignon, and San Giovese.

21. Use of a polyphenol in the manufacture of a nutriceutical, a food, a food supplement or a medicament for the prophylactic or therapeutic treatment of Endothelial dysfunction or the Metabolic Syndrome or other vascular sequellae wherein the polyphenol is obtained from grapes according to the method of claim 20.

22. A polyphenol for use in the manufacture of a nutriceutical, a food, a food supplement or a medicament for the prophylactic or therapeutic treatment of
